(19) United States
(12) Patent Application Publication
(10) Pub. No.: US 2025/0152650 A1
(43) Pub. Date: May 15, 2025

(54) COMPOSITIONS FOR TREATING SEA LICE IN SALMON AND RELATED METHODS

(71) Applicant: KEMIN INDUSTRIES, INC., DES MOINES, IA (US)

(72) Inventors: Mukkalil Rajalekshmi, Thiruverkadu (IN); Koenraad Frans Leon Meynen, Balen (BE)

(73) Assignee: KEMIN INDUSTRIES, INC., DES MOINES, IA (US)

(21) Appl. No.: 18/947,873

(22) Filed: Nov. 14, 2024

Related U.S. Application Data
(60) Provisional application No. 63/548,499, filed on Nov. 14, 2023.

(51) Int. Cl. A61K 36/534; A61K 31/05; A61K 31/122; A61K 36/71; A61P 33/14

(52) U.S. Cl. CPC A61K 36/534; A61K 31/05; A61K 31/122; A61K 36/71; A61P 33/14

(57) ABSTRACT

The present invention relates to compositions to treat sea lice in salmon, where such compositions do not impose safety hazards to aquatic life. According to one aspect, the present invention relates to methods of treating sea lice in salmon by administering a composition for treating sea lice in salmon, wherein the composition comprises monarda oil, alone or in combination with oregano oil with elevated levels of thymol, and optionally comprising one or more plant-based essential oil, where there is a synergistic effect with the combination of thymol and thymoquinone, and further where the composition is administered in an amount effective to treat copepodid stage and mobile staged sea lice. Another aspect of the present invention relates to a method of reducing copepodid and mobile-staged salmon lice activity by administering compositions containing an effective amount of monarda oil and oregano oil with elevated levels of thymol, alone or in combination with other plant-derived essential oils.

### CROSS-REFERENCE TO RELATED APPLICATIONS

The present application claims the benefit of priority to U.S. Provisional Patent Application No. 63/548,499, filed Nov. 14, 2023, entitled “COMPOSITIONS FOR TREATING SEA LICE IN SALMON AND RELATED METHODS,” the entire disclosure of which is hereby incorporated by reference in its entirety.

### BACKGROUND OF THE INVENTION

Sea lice or salmon lice (Lepeophteirus salmonis) are a significant concern in the salmon aquaculture industry, with an estimated global annual impact of 600 million to 1 billion USD. The increased resistance of lice to drugs and pesticides has forced the salmon aquaculture industry to develop treatments and mitigation steps. However, the treatments commercially available are not sufficiently effective, must be repeated with frequency, and cause undue stress on the salmon, resulting in substantial production losses. Accordingly, there is a long-felt need in the salmon industry for additional treatments and mitigation measures that can be implemented, as a replacement or supplement to existing treatments.

The number of wild Atlantic salmon has declined since the 1980's. The proliferation of L. salmonis from farmed salmon to wild salmon is thought to be one of the causes of poor marine survival of wild salmon. The governments of salmon-producing countries have put in force rules and regulations designed to decrease the risk of spreading sea lice from farmed to wild salmon. In Norway, during out-migration of juvenile salmon to sea, the maximum limit is 0.2 adult female lice per fish. Farmers are obliged to count the lice weekly and report these number along with the management measures that have been taken. In Scotland, the number of female L. salmonis per salmon, must be reported and once 6 female lice are found per fish, and triggers the requirement that measures be taken to reduce the number to 0.5 lice/fish or less. In Ireland, the trigger limit is 0.5 ovigerous lice per fish and in Canada it is 3 mobile lice per fish. The integrated management measures that can be taken include in-feed medicines, medicinal bath treatments, freshwater bath treatments, mechanical and biological treatments. All of these mitigation measures and treatments come with cost, not only for the treatment as such but also with environmental and side effect costs. For example, the mortality caused by a single treatment may range from 0.1% for a SLICE treatment, over 0.25% for a hydrolyzer (a water jet to separate lice from fish), 0.5% for fresh water treatment, thermolyzer (hot water) treatment or medicinal treatment, to 1% for a hydrogen peroxide treatment. Conventional lice management practices on a salmon farm typically include the use of skirts to create a physical barrier, cleaner fish (fish cultured with the salmon that eat the sea lice attached to the salmon) and one early treatment with SLICE, combined with one or two chemical or hydrogen peroxide treatments and four hydrolyzer or thermolyzer treatments during the salmon growing period. This combination causes, on average, a mortality of 2.6 to 3.2%, and in addition, these treatments result in stress and reduced feed intake and growth of the salmon. Indeed, addressing the issue of sea lice is generally regarded as one of the main challenges facing the salmon aquaculture industry today. The impact of sea lice cannot be understated, where towards the end of the growing period, salmon farmers might even prefer to bring the salmon to slaughter earlier than the full growing period in order to avoid exposing the salmon to an additional expensive round of sea lice treatment. Accordingly, there remains a long-felt need to provide innovative solutions to control, reduce, or eliminate the proliferation of sea lice. Compositions, such as dietary supplements, that could help to reduce the infection rate would be useful to decrease the number of lice on the fish and reduce the number of treatments salmon must endure through during the growing phase in sea water cages. This, in turn, will reduce the stress, improve well-fare and improve the overall growth and production of the salmon.

### SUMMARY OF THE INVENTION

The present invention relates to compositions to treat sea lice in salmon, where such compositions do not impose safety hazards to aquatic life. According to one aspect, the present invention relates to methods of treating sea lice in salmon by administering a composition for treating sea lice in salmon, wherein the composition comprises oregano oil, monarda oil, nigella oil, neem extract, thymol and thymoquinone in an amount effective to treat copepodid stage and mobile staged sea lice. Another aspect of the present invention relates to a method of reducing copepodid and mobile-staged salmon lice activity by administering compositions containing an effective amount of monarda oil and oregano oil, alone or in combination with other plant-derived essential oils.

### DETAILED DESCRIPTION OF THE FIGURES

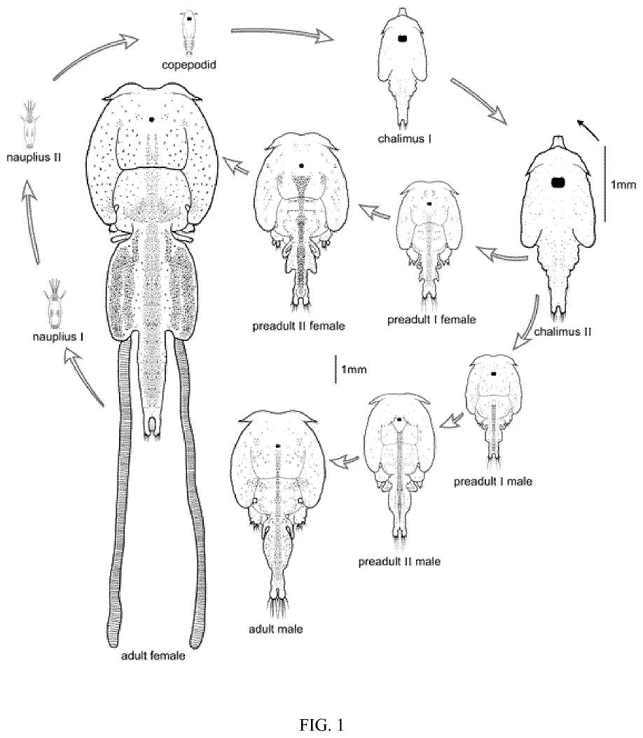

FIG. 1 depicts the life cycle of sea lice Lepeophteirus salmonis. (Source: Sea Lice Research Centre (University of Bergen)).

### DETAILED DESCRIPTION OF THE INVENTION

Plant derived components are potential sustainable source of antimicrobial chemotypes and remain untapped as potential solutions for controlling pathogens. The inventors have surprisingly identified a blend of plant-derived components capable of treating sea lice in salmon, where such compositions do not impose safety hazards to aquatic life.

Salmon lice or sea lice (Lepeophtheirus salmonis) are caligid copepods that are ectoparasites of salmon, feeding on mucus, skin and blood of the fish. Infestations can lead to physical damage, skin erosion, secondary infections, immunosuppression and chronic stress. With the intensive growth of salmon aquaculture, problems with sea lice infestations at salmon farms has become an increasingly critical issue, with a global estimated annual impact of 600 million to 1 billion USD¹. Numerous treatment options for sea lice are available and are used in conjunction within region-specific integrated pest management strategies. While drugs and pesticides continue to dominate these treatment regimens, a recent increase in the use of alternative treatments has emerged over the last decade; especially in Norway². Alternative treatments include the use of cleaner fish, plankton nets, deep water lighting, fresh water and mechanical and thermal treatments. A major driver of the switch to alternative therapies has been the development of multi-drug resistant strains of sea lice³. Though these alternative methods are often either not effective enough or cause high stress and elevated mortality, sea lice management strategies using chemical-based therapies as last resorts rather than treatments of choice are becoming more common.

The life cycle of L. salmonis consists of 8 stages, separated by moulting events (see FIG. 1). Free-living nauplii larvae hatch directly from the egg-string in the water¹¹. Both nauplius stages are planktonic and non-feeding. Second nauplius stage moults into the infectious copepodid stage. The copepodids use visual, mechanosensory and olfactory cues to find and identify a host¹². They typically attach to the fins, gills or scales of the fish by clasping with the second antennae. When molting to the first chalimus stage, they produce a frontal filament that punctures the epidermis and the lice remain sedentary until molting into the pre-adult stage. During both pre-adult and the adult stages, the lice are mobile and start grazing on the mucus and skin. Both copepodids and mobile stage lice were used in the study.

According to one aspect of the present invention, the researchers have surprisingly identified a statistically significant in vitro effect of monarda oil, alone or in combination with oregano oil, where the inventors observed a synergy between thymol and thymoquinone against infective copepodid stage and mobile staged sea lice. According to another aspect of the present invention, compositions containing an effective amount of oregano oil with elevated levels of thymol, alone or in combination with monarda oil resulted in complete inactivation of copepodids. Another aspect of the present invention relates to methods of using oregano oil with elevated levels of thymol and monarda oil with thymol and thymoquinone, to reduce copepodid and mobile-staged salmon lice activity.

According to at least one embodiment, the compositions of the present invention include monarda oil in an amount ranging from about 250 ppm to about 5000 ppm monarda oil on the feed. According to at least one embodiment, the compositions of the present invention include monarda oil in an amount of at least 250 ppm, and further comprises oregano oil in an amount ranging from about 250 ppm to about 5000 ppm oregano oil on the feed. In certain embodiments, the composition comprises extracts of monarda and oregano, for instance monarda oil and oregano oil, where monarda extract is present in an amount ranging from about 10 to 90 percent by weight and oregano extract is present in an amount ranging from about 10 to 90 percent by weight.

According to at least one embodiment, the compositions of the present invention include thymol in an amount ranging from about 50-80% of the oregano oil by weight, for instance at least 70% concentration, or at least 75% concentration. In certain embodiments, the thymol will be present in an amount ranging from about 150 ppm to about 5000 ppm, for instance about 175 ppm to about 3500 ppm. According to at least one embodiment, the compositions of the present invention contain thymoquinone in an amount ranging from about 40-60% of the monarda oil by weight, for instance at least 45% concentration, or at least 50% concentration. In certain embodiments, the thymoquinone will be present in an amount ranging from about 100 to about 3000 ppm, for instance about 125 ppm to about 2500 ppm.

In certain embodiments, the composition contains monarda and/or oregano oil with thymol in a total amount of at least 5% by weight. In alternative embodiments, the concentration of thymol is at least 7%. In alternative embodiments, the concentration of thymol is at least 7%. According to at least embodiment, the composition contains monarda oil and/or oregano oil with thymoquinone in a total amount of at least 3% by weight. In certain embodiments, the compositions contain thymoquinone and/or thymohydroquinone in an amount of at least 4.5% by weight.

According to at least one embodiment, the compositions of the present invention comprise a combination of thymol to thymoquinone in a ratio ranging from 1:10 to 10:1, such as, in a ratio ranging from 1:9 to 9:1.

According to at least one embodiment, the compositions of the present invention further comprise thymohydroquinone, where a combination of thymoquinone to thymohydroquinone in a ratio ranging from 20:1 to 30:1, such as, for instance 25:1.

According to certain embodiments, the compositions of the present invention are orally administered, for instance as a feed additive or component of the salmon diet. In an alternative embodiment, the compositions are topically administered, for instance as a dip or bath treatment.

According to certain embodiments, the compositions of the present invention are administered for a period of at least 10 days, at least 14 days, at least 21 days, or 30 days or more. According to at least one embodiment, the compositions are administered for about 14 days before and until harvest. In alternative embodiments, the compositions are administered for about 21 days before harvest. In alternative embodiments, the compositions are administered for about 30 days before harvest. In yet alternative embodiments, the compositions are administered for about 45 days before harvest.

In certain embodiments, the compositions are administered prophylactically, where even modest reduction rates would translate into substantial savings to the overall farming operation. For instance, in certain embodiments, the compositions are capable or reducing the proliferation of lice by at least 3%, 5%, 7%, 10% or more. In certain embodiments, the compositions are capable of reducing the number of attached lice by 14% or more. In certain embodiments, the compositions are capable of reducing the number of attached lice by 20% or more. In certain embodiments, the compositions are capable of reducing the number of attached lice by 40% or more.

According to certain embodiments, the compositions of the present invention may optionally comprise one or more essential oils, solvents, surfactants, co-surfactants, salts or monoglycerides, diglycerides and triglycerides of short chain fatty acids, and suitable carriers.

Persons of ordinary skill in the art will appreciate that other conventionally known applications fall within the scope of the present invention, including but not limited to dips or bath treatments.

The following examples illustrate the present invention and are not intended to be limiting.

### EXAMPLES

### Example 1

Lice source. Newly moulted copepodids were collected and transferred via temperature-controlled incubators and were used immediately. Lice numbers relative to water volume was quantified using a stereoscope to determine the number of lice/mL. Stock lice were diluted to achieve approximately 5 lice/mL for each well in the assay to contain approximately 15 lice for mobile lice (pre-adults and adults), collection was completed on the day prior to the bioassay and lice were incubated overnight to acclimate. Water temperature ranged between 6.0-9.6° C. during this holding phase.

Test products. In a first series of test against copepodids following extracts were used: Oregano oil Hi Thymol (HiT), Oregano oil Hi Carvacrol (HiC), Nigella oil, Monarda oil and extract of Azadirachta indica powder. All the oils were analyzed for content of thymol, carvacrol, thymoquinone (TQ) and thymohydroquinone (THQ). Results of that analysis is shown in Table 1. In the trials against mobile stage sea lice, Oregano oil HiC and neem extract were not tested and thymol and thymoquinone were included. These were then also tested against copepodids. All oils were well mixed immediately prior to use. Stock solutions were then prepared by mixing the oil with same volume of dimethylsulfoxide (DMSO) during 30 minutes, followed by addition of distilled water to obtain a 10% oil solution. When these were added in seawater, all oils went fully in solution, except for the Nigella oil which was not fully soluble and formed small globules that dispersed throughout the water. Thymol and thymoquinone stock solutions were prepared by dissolving 100 mg chemical in 7.5 ml DMSO and shake until fully dissolved. Then 7.5 ml water was added to obtain a 0.667% stock solution. As positive controls emamectin benzoate (EMB) and diallyl disulphide were used at concentrations expected to have 50% efficacy. EMB stock solution was prepared by dissolving 2.5 mg in 12.5 mL methanol and then 12.5 mL of distilled water was added and the solution was mixed. For diallyl disulfide (DD), 10 mg was dissolved in 1 mL DMSO before adding 9 mL of distilled H₂O. All stock solutions were diluted with sea water to obtain the test concentrations. DMSO and methanol were also tested as solvent controls.

TABLE 1
Concentration of actives found in the tested oils

| | | | | Thymohy- |
|---|---|---|---|---|
| | Thymol | Carvacrol | Thymoquinone | droquinone |
| Oil | (wt %) | (wt %) | (wt %) | (wt %) |
| Oregano oil HiT | 70.1 | 3.4 | nd | nd |
| Oregano oil HiC | trace | 66.8 | nd | nd |
| Nigella oil | nd | nd | trace | 2.5 |
| Monarda oil | nd | nd | 2.9 | 2.2 |

nd = not detected

Copepodid bioassay. As treatment group a combination of product and concentration is considered. For each treatment group, 3 mL was distributed into 12-well plates using a serological pipette. Stock compound concentrations were double the intended concentration, as a 1:1 mixture with seawater containing lice occurred at assay start. Wells received 3 mL of copepodid sea lice stock, with an intended concentration of 5 lice/ml (15 lice/well; 45 lice/treatment group). Once sea lice were added to their wells, time was recorded and assessments followed 24 hours later. Sea lice were held in an incubator between 10-12° C. for the bioassay. Seawater used for the assay was 32-34 ppt. Assessment of copepodid lice for “health/mobility status” was done using a binary classification of “live” and “dead”. Lice that respond to stimuli (i.e. brushing with forceps, phototaxis, and/or water bursts from a pipette) by swimming or crawling were considered live and those that did not respond were considered dead. Thus, mobility was the endpoint of the assay.

Mobile stages bioassay. Adult assays proceeded similarly to the copepodid assays; however, compounds were added directly to petri dishes rather than to 12-well plates, and no mixing of compound stock and lice stock was necessary and thus, nominal concentrations were added to each dish. Briefly, to start the assay, 15 mobile lice were placed in petri dishes containing seawater only. Just prior to the addition of compounds, the dish was swirled, and seawater poured off. All lice remaining attached to plastic were considered fit for the assay and those that fell out were discarded or placed back into the stock population. Replacement lice were added at this time to make up 15 individuals/dish, and further swirls as described above, were completed to ensure robustness. Thus, a minimum of 8 lice were present in each dish, with an average of 14 lice per dish. Three miscounts occurred that resulted in 16 lice/dish in three of the petri dishes included in the study. Compounds were added at the appropriate concentrations, the time recorded, and dishes incubated for 6 hrs. Assessments of mobile lice post-incubation were similar to the copepodid assay. Comments were made where appropriate for moribundity and weakness; however, for statistical analysis, live and weak classifications were grouped into “live” and moribund and dead were grouped as “dead”. Petri dishes were returned to the incubator (6-9° C.) for an additional 18 hours, at which time the 24-hour assessment was executed. In a follow-up trial studying the combination of thymol and thymoquinone, a 24-hour assessment was performed.

Statistics. Efficacy was determined for each compound and concentration combination with comparisons of treated and control groups using either probit or logistic regression models (p<0.05), depending on suitability. If a full range of efficacy was not determined and/or, the data distributed in a way that was not compatible with this analysis, a Dunnett's test (p<0.05) was used as a pairwise comparison following a significant Kruskal-Wallis Rank Sum Test (p<0.05).

Copepodid bioassay. Survival of copepodids from Huntsman Marine Science Centre in seawater and controls is shown in Table 2. The effect of exposure to test products on survival of these copepodids is shown in Table 3. Survival of copepodids of Norwegian origin after exposure to controls and test products is shown in Table 3 and 4.

TABLE 2
Mean proportion of survival (%) of copepodids of Canadian
origin in negative, positive and solvent controls
(n = 3 except for seawater control where n = 6)

| Treatment control | Survival (%) ± SD |
|---|---|
| Sea water control | 97.7 ± 3.7 |
| DMSO 1% | 86.6 ± 3.4 |
| DMSO 0.1% | 96.1 ± 6.8 |
| DMSO 0.01% | 92.2 ± 6.8 |
| DMSO 0.1% + canola oil 0.1% | 100.0 ± 0.0 |
| Methanol 1% | 80.6 ± 4.8 |
| Methanol 0.1% | 89.2 ± 8.3 |
| Methanol 0.01% | 95.4 ± 1.1 |
| EMB 50 ppb | 35.5 ± 2.1 |
| EMB 5 ppb | 50.7 ± 6.1 |
| EMB 0.5 ppb | 53.2 ± 10.2 |
| Diallyl disulfide 100 ppm | 17.4 ± 3.2 |
| Diallyl disulfide 10 ppm | 34.6 ± 6.4 |
| Diallyl disulfide 1 ppm | 86.3 ± 4.0 |
| Diallyl disulfide 100 ppb | 95.2 ± 4.6 |
| Diallyl disulfide 10 ppb | 93.6 ± 8.0 |
| Diallyl disulfide 1 ppb | 96.9 ± 2.7 |

TABLE 3
Mean proportion of survival (%) of copepodids or Canadian origin exposed
to different concentrations of test products during 24 hours.

| | | Mean portion of survival (% ± SD; n = 3) | | | | |
|---|---|---|---|---|---|---|
| | | Oregano | Oregano | Nigella | Monarda | Neem |
| Concentration | | oil HiT | oil HiC | oil | oil | extract |
| 1000 | ppm | 0.0 | 93.8 ± 5.8 | 0.0 ± 0.0* | 0.0 | Not tested |
| 100 | ppm | 0.0 | 98.1 ± 3.2 | 82.3 ± 9.9 | 0.0 | 79.3 ± 14.2 |
| 10 | ppm | 0.0 | 100.0 ± 0.0 | 58.1 ± 8.3* | 0.0 | 84.7 ± 8.3 |
| 1 | ppm | 0.0 | 97.6 ± 4.1 | 74.1 ± 5.0* | 0.0 | 89.3 ± 5.5 |
| 100 | ppb | 0.0 | 100.0± 0.0 | 66.9 ± 15.0* | 0.0 | 88.7 ± 3.7 |
| 10 | ppb | 0.0 | 100.0 ± 0.0 | 51.7 ± 13.2* | 0.0 | 91.9 ± 2.4 |

*Dunnett's test p < 0.05 in pairwise comparison with test group and control

TABLE 4
Mean proportion of survival (%) of copepodids of Norwegian
origin in negative, positive and solvent controls
after 24 hours exposure (Average ± SD; n = 3)

| Treatment | Survival (%) |
|---|---|
| Sea water control | 94.2 ± 1.5 |
| DMSO control 0.5% | 97.3 ± 2.3 |
| DMSO control 0.05% | 94.8 ± 3.4 |
| EMB 10 ppb | 0.0 ± 0.0 |
| EMB 1 ppb | 0.0 ± 0.0 |
| EMB 0.1 ppb | 0.0 ± 0.0 |

TABLE 5
Mean proportion of survival (%) of copepodids of Norwegian
origin exposed to different concentrations of test
products during 24 hours. (average ± SD; n = 3)

| | | | | Thymol + |
|---|---|---|---|---|
| | | Thymol | Thymoquinone | Thymoquinone |
| 10 | ppm | 56.1 ± 2.0* | 0.0 ± 0.0* | 0.0 ± 0.0 |
| 1 | ppm | 87.9 ± 3.9 | 75.6 ± 9.3* | 4.7 ± 4.2* |
| 100 | ppb | 84.4 ± 6.1 | 92.9 ± 9.1 | 92.5 ± 4.3 |
| 10 | ppb | 93.1 ± 9.3 | 100.0 ± 0.0 | 91.9 ± 11.1 |
| 1 | ppb | 100.0 ± 0.0 | 97.3 ± 2.5 | 94.3 ± 5.4 |
| 0.1 | ppb | 93.9 ± 4.4 | 98.6 ± 2.4 | 97.9 ± 3.6 |
| 0.01 | ppb | 98.7 ± 2.2 | 98.1 ± 3.2 | 97.2 ± 2.5 |

*Dunnett's test p < 0.05 in pairwise comparison with test group and control

Mobile stage bioassay. Survival of mobile stage sea lice (adults and pre-adults) in seawater and exposed to solvent controls, is shown in Table 6. The effect of exposure to test products on survival of these mobile stage lice is shown in Tables 7, 8 and 9.

TABLE 6
Mean proportion of survival (%, average ± SD) of
mobile stage sea lice in negative, positive and solvent
controls (n = 2 except for seawater control where n = 4)

| | Survival after | Survival after |
|---|---|---|
| Treatment control | 6 hours | 24 hours |
| Sea water control | 98.3 ± 3.3 | 91.5 ± 3.3 |
| Vegetable oil 1% | 100.0 ± 0.0 | 100.0 ± 0.0 |
| Methanol 0.2% | 93.3 ± 9.4 | 90.0 ± 14.1 |
| DMSO 1% | 90.0 ± 4.7 | 84.0 ± 3.8 |
| EMB 100 ppb | 96.7 ± 4.7 | 86.7 ± 9.4 |
| EMB 200 ppb | 56.7 ± 4.7 | 53.3 ± 18.9 |
| EMB 400 ppb | 36.7 ± 14.1 | 23.3 ± 4.7 |
| EMB 500 ppb* | | 46.9 ± 9.8 |
| EMB 5 ppm* | | 0.0 ± 0.0 |
| Diallyldisulfide 10 ppm | 100.0 ± 0.0 | 96.7 ± 4.7 |
| Diallyldisulfide 100 ppm | 96.2 ± 5.4 | 88.7 ± 5.8 |

*EMB concentrations used for follow up trial with Norwegian sourced copepodids

TABLE 7
Mean portion of survival (% ± SD; n = 2) of mobile lice after
6 hours exposure to different concentrations of test products

| | | Oregano | Nigella | Monarda | Neem |
|---|---|---|---|---|---|
| Concentration | | oil HiT | oil | oil | extract |
| 100 | ppm | 0.0 ± 0.0* | 94.4 ± 7.9 | 90.4 ± 4.1 | 75.7 ± 6.1 |
| 10 | ppm | 56.7 ± 4.7* | 100.0 ± 0.0 | 95.8 ± 5.9 | 97.2 ± 3.9 |
| 1 | ppm | 86.2 ± 0.7 | 90.9 ± 2.8 | 82.6 ± 1.1 | 77.6 ± 12.5 |
| 100 | ppb | 80.0 ± 9.4 | 95.5 ± 6.4 | 95.0 ± 7.1 | 96.9 ± 4.4 |
| 10 | ppb | 96.7 ± 4.7 | 86.2 ± 8.7 | 83.3 ± 14.1 | 96.2 ± 5.4 |
| 1 | ppb | 86.6 ± 4.0 | 95.8 ± 5.9 | 100.0 ± 0.0 | 97.2 ± 3.9 |

*Dunnett's test p < 0.05 in pairwise comparison with test group and control

TABLE 8
Mean portion of survival (% ± SD; n = 2) of mobile lice after
24 hours exposure to different concentrations of test products

| Concen | | Oregano | Nigella | Monarda | Neem |
|---|---|---|---|---|---|
| tration | | oil HiT | oil | oil | extract |
| 100 | ppm | 0.0 ± 0.0* | 70.9 ± 15.4 | 16.7 ± 23.6* | 75.7 ± 6.1 |
| 10 | ppm | 6.7 ± 9.4* | 95.8 ± 5.9 | 100.0 ± 0.0 | 97.2 ± 3.9 |
| 1 | ppm | 72.0 ± 18.2 | 90.9 ± 2.8 | 82.6 ± 1.1 | 77.6 ± 12.5 |
| 100 | ppb | 70.0 ± 14.1 | 91.3 ± 0.5 | 90.0 ± 14.1 | 96.9 ± 4.4 |
| 10 | ppb | 90.0 ± 4.7 | 92.3 ± 10.9 | 80.0 ± 18.9 | 96.2 ± 5.4 |
| 1 | ppb | 87.1 ± 0.6 | 81.4 ± 12.2 | 87.5 ± 17.7 | 97.2 ± 3.9 |

*Dunnett's test p < 0.05 in pairwise comparison with test group and control.

TABLE 9
Mean proportion of survival (%) of mobile sea lice exposed to different concentrations
of test products after 6 and 24 hours exposure (average ± SD; n = 2)

| | | | | | | Thymol + |
|---|---|---|---|---|---|---|
| | | Thymol | | Thymoquinone | | thymoquinone |
| Concentration | | 6 hours | 24 hours | 6 hours | 24 hours | 24 hours |
| 10 | ppm | 23.7 ± 6.9 | 0.0 ± 0.0* | 60.7 ± 5.1 | 0.0 ± 0.0* | 6.7 ± 9.4* |
| 1 | ppm | 92.8 ± 0.7 | 92.8 ± 0.7 | 100.0 ± 0.0 | 83.7 ± 14.7 | 41.5 ± 26.1* |
| 100 | ppb | 97.1 ± 4.2 | 97.1 ± 4.2 | 91.7 ± 2.4 | 91.7 ± 2.4 | 100.0 ± 0.0 |
| 10 | ppb | 95.8 ± 5.9 | 88.1 ± 5.0 | 81.7 ± 5.4 | 87.1 ± 5.4 | 92.9 ± 0.0 |
| 1 | ppb | 100.0 ± 0.0 | 96.2 ± 5.4 | 93.3 ± 9.4 | 93.3 ± 9.4 | 100.0 ± 0.0 |

*Dunnett's test p < 0.05 in pairwise comparison with test group and control

Copepodid sensitivity to solvents and positive controls. All groups of solvent controls used in this study for L. salmonis copepodids of Canadian origin resulted in proportions of survival exceeding, on average, 85%. The exception to this was the 1.0% methanol group; however, this group was not used in the study for comparative purposes, no test solution had more than 0.1% methanol. Survival in seawater controls varied between 91.2%-100% while complete survival was observed in controls containing 0.1% DMSO with 0.1% canola oil. Some impacts on survival were noted for 0.1% and 0.01% DMSO groups, which varied between 84.2-100% survival in replicate test wells. Thus, it is clear that some baseline mortality occurred throughout the 24-hr assay, as expected, and that baseline copepodid survival in any test well could be expected to result in proportions between 85-100%, averaging well above 90% for most control types. Positive controls for copepodids included emamectin benzoate (EMB) and diallyl disulfide (DD). In-feed administration of EMB was the treatment of choice for veterinarians and farmers from 1999-2008, before EMB-resistant strains of L. salmonis became widespread throughout the Atlantic¹³⁻¹⁷. However, copepodid larvae are far more sensitive to EMB compared with mobile stages, and thus a dilution set between 0.5-50 ppb EMB was applied, resulting in similar levels of survival in each concentration (35.5-53.2% survival; Table 2); all of which significantly differed from the 0.1% methanol control (p<0.05; Dunnett's pairwise comparison test). It is noteworthy that the actual concentration of methanol in the highest (50 ppb) EMB group was 0.025%, but the 0.1% methanol control was used as a comparator. In a follow-up trial with copepodids sourced in Norway all tested EMB concentration, ranging between 10 and 0.1 ppb, resulted in no survival. These copepodids are derived from a strain that has no resistance against EMB, while the sea lice in Bay of Fundy are known to have built resistance against EMB.

When diallyldisulfide (DD) was applied, only the two highest concentrations (100 ppm and 10 ppm) were effective in reducing survival of copepodid sea lice compared to 1% DMSO controls. Rates of survival were 17.4% for 100 ppm DD and 34.6% for 10 ppm. Importantly, the 100 ppm group had 1% DMSO, which was shown to have a slight effect on survival and thus one could speculate an interaction effect of DMSO and DD in this group. However, the 10 ppm group with only 0.1% DMSO shows a clear, singular, impact of DD on louse mobility based on complete survival of lice exposed to 0.1% DMSO. Treatment such as DD are often described for their repellent effects, at concentrations much lower than those showing effects here¹⁸ (18). The use of garlic and its components, for sea lice control has been explored previously, showing reduction in lice levels on salmon when delivered in feed for some studies¹⁹. Similarly, bags of garlic and onion were suspended in cages in the early 1990s in an attempt to repel free-swimming stages of lice²⁰. Pesticides were however approved and used widely by this time and thus implementation and collection of efficacy data for this repellant was sparse. Purified diallyl sulfide and diallyl disulfide masked copepodid sea lice attraction to hosts or alpha-isophorone, a salmon semiochemical, conditioned seawater at extremely low concentrations (10 parts per trillion) in a previous experiment¹⁸, suggesting repellant activity of these compounds. Thus, the contents of each test phytochemical, and the respective active ingredients within, are key to increasing efficacy against lice, highlighting the importance of synthesis, extraction, and purification processes for these oils to optimize the inclusion of active ingredients.

Copepodid Sensitivity to Test Compounds. All oil-based test compounds, except for oregano oil HiC, significantly reduced the survival of copepodid L. salmonis. Given no mortality was observed in controls with inclusions of 0.1% canola oil paired with 0.1% DMSO, the effects on survival from oregano oil HIT, nigella oil and monarda oil, were clearly due to active ingredients in the oils. For both oregano oil HiT and monarda oil, no survivors were observed at any concentration, suggesting the minimum lethal concentration (MLC) is at 10 ppb or lower. They were not statistically compared to controls due to complete efficacy observed at all concentrations. For nigella oil, only the highest concentration of 0.1% was completely lethal. Survival after 24 hr treatment with 0.01% nigella oil did not yield a statistically significant effect on survival, but significant reductions in survival were observed for all remaining concentrations (10 ppm-10 ppb). Mean proportions of survival between 51.7-74.1% were observed for these lower concentrations of Nigella oil, though the response was not dose-dependent. Finally, neem powder extract did not display statistically significant efficacy against L. salmonis copepodid survival or mobility. Treatment of copepodids with 10 ppb oregano oil HiT or monarda oil, resulted in higher mortality than the highest tested concentration of EMB (50 ppb) or DD (100 ppm).

Given the strong results oregano oil HiT and monarda oil, it was decided to do a follow up study with thymol and thymoquinone, active ingredients present in these oils. Thymol and thymoquinone were assayed at concentrations between 10 ppm and 0.1 ppb separately, and combined in a 1:1 ratio. Thymoquinone and a combination of thymol and thymoquinone at 10 ppm resulted in 100% lethality. However, 1 ppm of thymoquinone did not result in a biologically significant reduction in survival. Likewise, none of the concentrations of thymol at less than 1 ppm impacted lice, with only 43.9% of lice mortality at 10 ppm thymol. Interestingly, the combination of thymol and thymoquinone at 1 ppm (500 ppb each) resulted in lice mortality of 95.3%, displaying clear synergy between the two compounds in copepodid lice.

Mobile-stage Lice Sensitivity to solvents and controls. All groups of solvent controls used in this study for mobile stage lice resulted in proportions of survival exceeding, on average, 90%. The exception to this was the 1.0% DMSO with 84% survival; however, no test solution had more than 0.1% DMSO. Exposure to 10 and 100 ppm diallyl disulphide (DD) did not result in different survival compared to control. Exposure to 100 ppb did not impact survival of the mobile stage lice, while exposure to 5 ppm resulted in complete inactivation of the lice. Tested intermediate concentrations of 200 to 500 ppm resulted in partial inactivation ranging on average between 46.7 and 76.7%. These results are in line with published EC50 results obtained with lice collected from different sources in British Columbia, Canada²¹.

Mobile stage lice Sensitivity to Test Compounds. Given the results obtained with first trial with copepodids, it was decided to remove oregano oil HiC from the test groups, and to include test groups with thymol and thymoquinone. As with the copepodid assays, Oregano oil HiT and Monarda oil were most efficacious against L. salmonis in this study. Only 100 ppm Oregano oil HiT was completely lethal, while 10 ppm left two survivors of 30 total challenged lice. The estimated half-maximal effective concentrations (EC50) at 6 hrs post treatment administration with Oregano oil HIT was 11 ppm while after 24 hrs of treatment, the EC50 was estimated at 2.9 ppm. Though average survival in 1 ppm and 100 ppb Oregano oil HiT was near 70%, suggesting a minor effect, the results were not statistically significant and high levels of variation between duplicate dishes were observed for both groups. For 1 ppm Oregano oil HIT, 19 of 29 lice survived 24 hr treatment, while at 100 ppb, 19 of 30 lice survived treatment. It is clear that a greater number of lice died as a result of these treatments compared with controls, however, the variation observed between replicates requires further investigation and may be linked to genotype. The highest concentration of Monarda oil tested, 100 ppm, showed efficacy with some survival still observed (5 out of 30 lice survived treatment). There were no impacts on survival or mobility in concentrations of 10 ppm or below. Nigella oil overall was not efficacious against adults. As with the copepodid assays, neem powder extract was not efficacious against mobile staged lice.

Thymol and thymoquinone were completely lethal after 24 hours exposure to 10 ppm, a combination of thymol and thymoquinone resulted an average survival of 6.7% at 10 ppm and 45.1% at 1 ppm. Exposure to 1 ppm thymol or thymoquinone was not significantly different to control, while the combination was. This again demonstrates the synergy between the two chemicals.

In conclusion, the inventors have identified the capability of treating sea lice in salmon by administering a blend of plant-derived essential oils, including but not limited to the use of oregano oil with elevated thymol levels (HiT), monarda oil, thymol and thymoquinone, in water to reduce copepodid and mobile-staged sea lice activity.

Although known administration routes would fall within the scope of this disclosure, in at least one embodiment feed applications are preferred due to labor intensity and cost of bath treatments, as well as the associated stress for the fish and environmental concerns. Feeding monoterpenes to fish can result in their presence in the skin mucus, as reported with carvacrol in juvenile chum salmon²² and 1,8-cineole in common carp²³. These molecules were found in the skin mucus at ppb and ppm level after dietary administration. It is important to note that uptake and distribution of active compounds by sea lice differs from the bioassay model employed here (absorption through respiration) compared to the parasitic system, where compounds are absorbed through the digestive tract during feeding and through contact with skin/mucous of hosts for the entire parasitic phase of the lifecycle. It is unknown how this difference translates to in-feed administration of the oils tested here; however, in the case of emamectin benzoate (EMB), an in-feed drug targeting sea lice, similar concentrations (1-10 ppb) in salmon tissues (in vivo) and 24 hr bioassay baths (in vitro) appear to be lethal to sensitive copepodid lice^{9,10} (CATC studies).

The plant-based essential oils tested in this study likely contain a combination of active ingredients with differing levels of activity against lice and there may be possibilities for modifications that would increase efficacy. The inclusion of thymoquinone or the combination of thymol and thymoquinone into feed formulations might be a more economical process with greater uptake efficacy, however differences in activity between pure thymol and Oregano oil HiT or between thymoquinone and Monarda oil, suggest that other components present in the plant-based oils have an effect on the activity compared to the molecules deliver alone.

The results also indicate that there is an unexpected synergistic effect of thymol and thymoquinone offering promising possibilities for reducing sea lice infection in salmon. Further, the data indicates that the application of oregano oil HiT, Monarda oil, thymol and thymoquinone may potentially disrupt host attachment and parasitism that will warrant further investigation.

In addition to a direct effect on activity of sea lice, the phytochemicals can also decrease sea lice infection by disturbing the host-seeking behavior of the sea lice¹⁸ and by stimulating the immune system of the fish making them more resistant to lice infections²⁴⁻²⁶. An in vivo trial with in-feed administration of these oils and chemicals, is planned to better inform on appropriate dosing and administration regimens for future work. Important experimentation targeting the pharmacokinetic properties of these oils, including uptake (e.g. palatability) and excretion in the mucus, will also be beneficial alongside testing efficacy to determine therapeutic indices and economic viability of the treatments.

### Example 2

The current study aimed to evaluate the potential of oregano oil, Monarda oil, thymol, and thymoquinone individually as feed additives to control sea lice infection in Atlantic salmon (Salmo salar).

At study initiation, Atlantic salmon smolts were divided into tanks of 120 L volume, 40 fish per tank were used, 3 replicate tanks per treatment. The tanks were installed within a recirculation system that employs biofilters, UV sterilization, sand filters, bag filters (100 μm) while adding ca. 8-10 L/min of saltwater from a well as make-up water. The study diets were prepared by top-coating a Salmonid base diet with each treatment ingredient and then herring oil if required.

The treatments for the study are summarized in Table 10. Oregano oil was used as a source of thymol, Monarda fistulosa was used as a source of thymoquinone. SLICE (emamectin benzoate) was used as a positive control.

TABLE 10
Study Treatments

| Study Diet | Study Diet ID |
|---|---|
| Control Diet | CONTROL |
| Oregano Oil (2000 ppm) | O-LD |
| Oregano Oil (5000 ppm) | O-HD |
| Monarda fistulosa oil (2000 ppm) | M-LD |
| Thymol (1000 ppm) | T |
| Thymoquinone (500 ppm) | TQ |
| SLICE ® (10 ppm) | S |

All tanks were provided with a 1% of tank biomass daily aliquot of their study diet based on the bulk weight assessments. Fish were fed their respective diet during 3 weeks, before being challenged with copepodid lice.

Sea lice were sourced from eggs collected aboard a treatment vessel that utilizes water pressure to remove sea lice from fish. The lice removed from the fish were captured on large paper filters where they were then collected. The sea lice egg strings raised to the copepodid stage in filtered sea water. Once developed into copepodids, the lice were transported the trial facility where they were immediately assessed for viability and used to conduct a sea lice challenge.

To conduct the challenge, the sea lice were consolidated into a single larval pool. To increase larval activity, study fish tank water was used to adjust the larval pool volume to 10 L. The larval pool was gently stirred to raise the larvae onto the water column and five replicate 1 mL samples were taken with a volumetric pipette. The five water samples were analyzed using a Bogorov tray and stereoscope to count the number of active copepodids present per sample. The average number of active larvae per mL was calculated to be 16.6 copepodids per mL and this number was used to calculate the dosage necessary to infect each tank with approximately 140 lice per fish. Once the necessary larval pool volume was calculated for each tank, the larval pool was gently mixed, and a graduated cylinder was used to measure out tank aliquots.

To initiate the lice challenge, the water flow was shut off to all tanks and the volume of water within each tank was lowered by approximately 15-30%. This was done to increase fish density and to avoid lice escaping via the upper tank overflow drain. The lice were added to all tanks simultaneously and the transfer bottles were rinsed to ensure all larvae entered the tanks. The fish remained in static water for 1 hour and 16 minutes before the water flow was restored to the tanks. During the exposure period, the tanks were supplemented with oxygen to ensure dissolved oxygen stayed above 80% saturation.

At six- and seven-days post-infection (dpi), a two-day sample event occurred with 10 fish sampled per tank. At day 6 dpi, two replicate tanks from each study diet were sampled and on day 7 dpi the remaining replicate tank from each study diet was sampled. The focus was to count the number of copepodid and chalimus lice on the fish as a measure of how well the study diets prevented initial infection. Lice counts were completed by first euthanizing the fish with tricaine methanesulfonate (TMS) in individual containers. Once euthanized, the body of each fish was examined for chalimus and copepodids by eye. The opercula, gill arches, and fins were removed for examination under stereoscope. The euthanasia containers were examined by eye and any detached lice recovered were included in the body count for that fish. All the lice found in the gills, and on the opercula, fins, and body, were summed to obtain the total number of lice per fish. The results of this count are shown in below Table 11.

TABLE 11
Reduction in Mean Number of Lice per treatment.

| | Mean | | Reduction | Reduction |
|---|---|---|---|---|
| | Number | Standard | Versus | Versus |
| Study Diet | of Lice | Deviation | Control | SLICE |
| Control Diet | 73.8 | 27.4 | — | 2.2% |
| M-LD | 56.8 | 41.9 | 23.0% | 24.7% |
| O-HD | 41.6 | 13.8 | 43.7% | 44.9% |
| O-LD | 63.3 | 33.5 | 14.3% | 16.2% |
| SLICE | 75.5 | 19.7 | −2.3% | — |
| Thymol | 86.5 | 15.0 | 1.2% | −14.6% |
| Thymoquinone | 59.1 | 19.2 | 1.7% | 21.7% |

The researchers observed that the addition of 2000 ppm monarda oil or 5000 ppm oregano oil (rich in thymol), resulted in a significant decrease in number of sea lice found on the fish. The study results confirmed that Monarda oil and oregano oil appears to have great potential as a prophylactic treatment for reducing the severity of sea lice infections in Atlantic salmon.

Having described the invention with reference to particular compositions, theories of effectiveness, and the like, it will be apparent to those of skill in the art that it is not intended that the invention be limited by such illustrative embodiments or mechanisms, and that modifications can be made without departing from the scope or spirit of the invention, as defined by the appended claims. It is intended that all such obvious modifications and variations be included within the scope of the present invention as defined in the appended claims. The claims are meant to cover the claimed components and steps in any sequence which is effective to meet the objectives there intended, unless the context specifically indicates to the contrary.

It should be further appreciated that minor dosage and formulation modifications of the composition and the ranges expressed herein may be made and still come within the scope and spirit of the present invention.

It is also to be understood that the formulations and processes illustrated in the attached drawings, and described in the following specification are simply exemplary embodiments of the inventive concepts defined in the appended claims. Hence, specific dimensions and other physical characteristics relating to the embodiments disclosed herein are not to be considered as limiting, unless the claims expressly state otherwise. Where a range of values is provided, it is understood that each intervening value, to the tenth of the unit of the lower limit unless the context clearly dictates otherwise, between the upper and lower limit of that range, and any other stated or intervening value in that stated range, is encompassed within the scope of the present disclosure. The upper and lower limits of these smaller ranges may independently be included in the smaller ranges, and are also encompassed within the scope of the present disclosure, subject to any specifically excluded limit in the stated range. Where the stated range includes one or both of the limits, ranges excluding either or both of those included limits are also included in the scope of the present disclosure. All ranges and parameters, including but not limited to percentages, parts, and ratios, disclosed herein are understood to encompass any and all sub-ranges assumed and subsumed therein, and every number between the endpoints. For example, a stated range of “1 to 10” should be considered to include any and all sub-ranges beginning with a minimum value of 1 or more and ending with a maximum value of 10 or less (e.g., 1 to 6.1, or 2.3 to 9.4), and to each integer (1, 2, 3, 4, 5, 6, 7, 8, 9, 10) contained within the range. In this specification and the appended claims, the singular forms “a,” “an” and “the” include plural reference unless the context clearly dictates otherwise. All combinations of method steps or process steps as used herein can be performed in any order, unless otherwise specified or clearly implied to the contrary by the context in which the referenced combination is made.

To the extent that the terms “includes” or “including” or “have” or “having” are used in the specification or the claims, it is intended to be inclusive in a manner similar to the term “comprising” as that term is interpreted when employed as a transitional word in a claim. Furthermore, to the extent that the term “or” is employed (e.g., A or B) it is intended to mean “A” or “B” or both “A” and “B”. When the Applicant intends to indicate “only A or B but not both” then the term “only A or B but not both” or similar structure will be employed. Thus, use of the term “or” herein is the inclusive, and not the exclusive use. Also, to the extent that the terms “in” or “into” are used in the specification or the claims, it is intended to additionally mean “on” or “onto.” In this specification and the appended claims, the singular forms “a,” “an” and “the” include plural reference unless the context clearly dictates otherwise.

The foregoing description has been presented for the purposes of illustration and description. It is not intended to be an exhaustive list or limit the invention to the precise forms disclosed. It is contemplated that other alternative processes and methods obvious to those skilled in the art are considered included in the invention. The description is merely examples of embodiments. It is understood that any other modifications, substitutions, and/or additions may be made, which are within the intended spirit and scope of the disclosure. From the foregoing, it can be seen that the exemplary aspects of the disclosure accomplish at least all of the intended objectives.

### REFERENCES

1. Intrafish Media. 2019 Business intelligence: The secret lives of sea lice. Intrafish.com. [Online] Jul. 3, 2019. https://www.intrafish.com/aquaculture/business-intelligence-the-secret-lives-of-sea-lice/2-1-632875.
2. Overton, Kathy, et al. 2019. Salmon lice treatments and salmon mortality in Norwegian aquaculture: a review. Reviews in Aquaculture. November 2019, November 2019, Vol. 11, 4, pp. 1398-1417.
3. Aaen, Stian Mørch, et al. 2015 Drug resistance in sea lice: a threat to salmonid aquacultur. Trends in Parasitology. Jan. 29, 2015, Vol. 31, pp. 72-81.
4. Poley, J, et al. 2013. Combinatorial effects of administration of immunostimulatory compounds in feed and follow-up administration of triple-dose SLICE® (emamectin benzoate) on Atlantic salmon, Salmo salar L., infection with Lepeophtheirus salmonis. 3, March 2013, Journal of Fish Diseases, Vol. 36, pp. 299-309.
5. Purcell, S. L., et al. 2013 CpG inclusion in feed reduces sea lice, Lepeophtheirus salmonis, numbers following re-infection. 3, March 2013, Journal of Fish Diseases, Vol. 36, pp. 229-240.
6. Covello, J. M., et al 2012 Effects of orally administered immunostimulants on inflammatory gene expression and sea lice (Lepeophtheirus salmonis) burdens on Atlantic salmon (Salmo salar), Nov. 5, 2012, Aquaculture, Vols. 366-367, pp. 9-16.
7. Sutherland, Ben J. G., et al. 2017. Host-parasite transcriptomics during immunostimulant-enhanced rejection of salmon lice (Lepeophtheirus salmonis) by Atlantic salmon (Salmo salar). Jun. 8, 2017, FACETS, Vol. 2, pp. 477-495.
8. Bricknell, Ian and Dalmo, Roy A. 2005. The use of immunostimulants in fish larval aquaculture. Fish and Shellfish Immunology, Vol. 19, pp. 457-472.
9. Stone, J., et al. 2000 Commercial trials using emamectin benzoate to control sea lice Lepeophtheirus salmonis infestations in Atlantic salmon Salmo salar. 2, Jun. 19, 2000, Diseases of Aquatic Organisms, Vol. 41, pp. 141-149.
10. Poley, Jordan D., Igboeli, Okechukwu O. and Fast, Mark D. 2015. Towards a consensus: Multiple experiments provide evidence for constitutive expression differences among sexes and populations of sea lice (Lepeophtheirus salmonis) related to emamectin benzoate resistance. Nov. 1, 2015, Aquaculture, Vol. 448, pp. 445-450.
11. Aaen, Stian Mørch, 2015. Chemotherapeutants against salmon lice Lepeophtheirus salmonis-screening of efficacy. Adamstuen: Norwegian University of Life Sciences, 2016. Thesis number 2016:80. ISBN 978-82-575-1978-0/ISSN 1894-6402.
12. Fields, D M, Skiftesvik, A B and I, Browman H. 2017 Behavioural responses of infective-stage copepodids of the salmon louse (Lepeophtheirus salmonis, Copepoda:Caligidae) to host-related sensory cues. Jun. 26, 2017, Journal of Fish Diseases, Vol. 00, pp. 1-10.
13. Aaen, Stian Mørch, et al. 2015. Drug resistance in sea lice: a threat to salmonid aquaculture. February 2015, Trends in Parasitology, Vol. 31, pp. 72-81.
14. Igboeli, Okechukwu O, Burka, John F and Fast, Mark D. 2014. Sea lice population and sex differences in P-glycoprotein expression and emamectin benzoate resistance on salmon farms in the Bay of Fundy, New Brunswick, Canada. June 2014, Pest Management Science, Vol. 70, pp. 905-914.
15. P. G., Jones, et al. 2013. Detection of emamectin benzoate tolerance emergence in different life stages of sea lice, Lepeophtheirus salmonis, on farmed Atlantic salmon, Salmo salar L. 3, March 2013, Journal of Fish Diseases, Vol. 36, pp. 209-220.
16. Sutherland, Ben J. G., et al. 2015. Transcriptomic responses to emamectin benzoate in Pacific and Atlantic Canada salmon lice Lepeophtheirus salmonis with differing levels of drug resistance. February 2015, Evolutionary Applications, Vol. 8, pp. 133-148.
17. Carmichael, Stephen N, et al. 2013. Salmon lice (Lepeophtheirus salmonis) showing varying emamectin benzoate susceptibilities differ in neuronal acetylcholine receptor and GABA-gated chloride channel mRNA expression. Jun. 18, 2013, BMC Genomics, Vol. 14.
18. O'Shea, B, et al. 2017. Disruption of host-seeking behaviour by the salmon louse, Lepeophtheirus salmonis, using botanically derived repellents. April 2017, Journal of Fish Diseases, Vol. 40, pp. 495-505.
19. K, Boxaspen and JC, Holm. 1991. New biocides used against sea lice compared to organo-phosphorous compounds. Dublin, Ireland: European Aquaculture Society. Aquaculture and the Environment: reviews of the International Conference Aquaculture Europe '91. 90-71625-10-9.
20. Roth, M., Richards, R. H. and I, C. Sommerville. 1993. Current practices in the chemotherapeutic control of sea lice infestations in aquaculture: a review. 1, 1993, Journal of Fish Diseases, Vol. 16, pp. 1-26.
21. Saksida, S M, et al. 2013. Use of Atlantic salmon, Salmo salar L., farm treatment data and bioassays to assess for resistance of sea lice, Lepeophtheirus salmonis, to emamectin benzoate (SLICE®) in British Columbia, Canada. 2013, Journal of Fish Diseases, Vol. 36, pp. 515-520.
22. Mizuno, S., et al. 2018. Effects of dietary supplementation with oregano essential oil on prevention of the ectoparasitic protozoans Ichthyobodo salmonis and Trichodina truttae in juvenile chum salmon Oncorhynchus keta. September 2018, Journal of Fish Biology, Vol. 93, pp. 528-539.
23. Zoral, M. A., et al. 2018. Toxicological effects and pharmacokinetics of rosemary (Rosmarinus officinalis) extract in common carp (Cyprinus carpio). October 2018, Aquaculture, Vol. 495, pp. 955-960.
24. Holm, Helle Jodaa, et al. 2016. Dietary phytochemicals modulate skin gene expression profiles and result in reduced lice counts after experimental infection in Atlantic salmon. 271, 2016, Parasites & Vectors, Vol. 9.
25. Jensen, L. B., et al. 2015. Reducing sea lice (Lepeophtheirus salmonis) infestation of farmed Atlantic salmon (Salmo salar L.) through functional feeds. December 2015, Aquaculture Nutrition, Vol. 21, pp. 983-993.
26. Firmino, Joana P., et al. 2021. Phytogenic Bioactive Compounds Shape Fish Mucosal Immunity. Frontiers in Immunology, Vol. 12. Article 695973, 2021.

1. A method of treating sea lice in salmon by administering a composition comprising an extract of monarda in an amount effective to reduce the number of the sea lice attached to the salmon.

2. The method of claim 1, wherein the composition comprises monarda oil in an amount effective to reduce the number of the sea lice attached to the salmon.

3. The method of claim 1, wherein the composition further comprises an extract of oregano.

4. The method of claim 1, wherein the composition further comprises an extract of oregano oil with thymol present in an amount of about 40% or more.

5. The method of claim 1, wherein the composition comprises one or more plant-based essential oil.

6. The method of claim 35, wherein the one or more plant-based essential oil is selected from the group consisting of monarda oil, oregano oil and nigella oil.

7. The method of claim 1, wherein the composition is orally administered to the salmon.

8. The method of claim 1, wherein the composition is topically administered to the salmon.

9. A method of treating sea lice in salmon by administering a composition comprising thymoquinone in an amount effective to upregulate wound healing gene expression or reduce the number of the sea lice attached to the salmon.

10. The method of claim 69, wherein the thymoquinone is derived from one or more plant-based essential oil.

11. The method of claim 69, wherein the thymoquinone is derived from monarda or nigella.

12. The method of claim 19, wherein the composition further comprises an extract of oregano oil with thymol present in an amount of about 40% or more.

13. The method of claim 19, wherein the composition comprises one or more plant-based essential oil selected from the group consisting of monarda oil, oregano oil and nigella oil.

14. The method of claim 69, wherein the composition is orally administered to the salmon.

15. The method of claim 69, wherein the composition is topically administered to the salmon.

16. A method of reducing copepodid and mobile-staged salmon lice activity by administering compositions containing an effective amount of monarda oil and oregano oil with elevated levels of thymol.

17. The method of claim 1116, wherein the composition further comprises one or more plant-derived essential oils.

18. The method of claim 116, wherein the composition further comprises an extract of oregano oil with thymol present in an amount of about 40% or more.

19. The method of claim 1116, wherein the composition is orally administered to the salmon.

20. The method of claim 1116, wherein the composition is topically administered to the salmon.
